(19) United States
(12) Patent Application Publication
(10) Pub. No.: US 2018/0221842 A1
(43) Pub. Date: Aug. 9, 2018

(54) MULTI-STAGE REACTOR AND SYSTEM FOR MAKING METHANOL IN A ONCE-THROUGH PROCESS AND METHODS THEREFOR

(71) Applicant: PRIMUS GREEN ENERGY INC., Hillsborough, NJ (US)

(72) Inventors: Eli GAL, Sunnyvale, CA (US); Robert M. KOROS, Westfield, NJ (US); Benjamin MOSKOWITZ, Monmouth Junction, NJ (US); Arnold KELLER, Henderson, NV (US)

(21) Appl. No.: 15/750,027

(22) PCT Filed: Aug. 9, 2016
(86) PCT No.: PCT/US2016/046113

§ 371 Date: Feb. 2, 2018

Related U.S. Application Data
(60) Provisional application No. 62/202,999, filed on Aug. 10, 2015.

(51) Int. Cl. B01J 8/04; B01J 8/06; C07C 29/152; C07C 31/04

(52) U.S. Cl. CPC B01J 8/0449; B01J 8/065; B01J 8/067; C07C 29/152; F28D 2021/0022; B01J 2208/00274; B01J 2208/0053; B01J 2208/065; C07C 31/04

(57) ABSTRACT

The present invention relates to a multi-stage, single reactor and system for making methanol for synthesis gas (syngas). In particular, the reactor contains a shell and tube reactor that is divided at its top and bottom heads into a plurality vertical, isolated compartments. The associated compartments and tubes form a stage of the reactor. The raw syngas is fed to the first stage, and unreacted syngas from the first stage is fed to the second stage subsequent stages. Between each stage, the product, methanol and water, is removed from the reaction mixture before sending the unreacted syngas to the subsequent stage. The reactor allows for high conversion of synthesis gas to methanol in a once-through process, without requiring recycling of unreacted synthesis gas.

### CROSS-REFERENCE TO RELATED APPLICATION

This application claims the priority of U.S. Provisional Patent Application No. 62/202,999, filed Aug. 10, 2015, which is incorporated herein by reference.

### FIELD OF THE INVENTION

The present invention relates to a multi-stage, single reactor and system for making methanol from synthesis gas. In particular, the reactor contains a shell and tube vessel that is divided at its top and bottom heads into several vertical compartments. The reactor allows for high conversion of synthesis gas to methanol in a once-through process, without requiring recycling of unreacted synthesis gas.

### BACKGROUND

Synthesis gas (syngas) typically contains CO, CO₂ and H₂ as the active species and CH₄ and N₂ as inert species and can be used to make methanol in a syngas-to-methanol process. The syngas can be made, e.g., from steam methanol reforming or auto-thermal reforming. The syngas typically contains excess H₂ to achieve high conversion of the COx species (CO and CO₂). The syngas-to-methanol process involves three reactions that are all exothermic and reversible as follows:

CO+2H₂<---->CH₃OH+Heat 1)

CO₂+3H₂<---->CH₃OH+H₂O+Heat 2)

CO+H₂O<---->CO₂+H₂+Heat 3)

To enhance the syngas-to-methanol reactions, a catalyst may be used, e.g., a metal catalyst, typically copper/zinc based catalyst.

In current state of the art syngas-to-methanol processes, the reactor is cooled to maintain an optimal temperature in the range of 210-270° C. and operates at a pressure typically greater than about 70 bar to achieve high COx conversion per pass. Depending on pressure, temperature and syngas composition, typical per pass conversion of the COx species is in the range of 30-70% which is too low for a practical commercial process. That conversion limit exists due to the reversible nature of the reactions where the rate of production of methanol competes with the rate of its decomposition when its concentration increases. The equilibrium limit can be overcome by removing the products and allowing the reactions to continue.

In a typical state of the art methanol plant, the syngas passes through the reactor and exits at a point approaching chemical equilibrium. The proximity of the exit stream to chemical equilibrium is determined by the operating conditions and the size of the reactor, which is typically designed for an economical optimum. The gas exiting the reactor is cooled; and the products, methanol and water, are condensed, separated from the gas, and removed from the process. The remaining gas, which contains unreacted CO, CO₂ and excess H₂ species and inerts from the feed, is recycled back to the reactor, typically after mixing with fresh syngas. By removing the products and recycling the residual gas high overall COx conversion, typically 95-98% can be achieved. Achieving high COx conversion efficiency requires high recycle rate in the range of 3-7 times mole recycle to mole of feed. While high conversion efficiency can be achieved by recycling the syngas, it comes at high costs as follows:
large reactor to handle large quantities of gas relative to the feed;
large recycle compressor to recycle the gas to the reactor inlet;
large heat exchangers to handle much larger volumes of gas relative to the feed and much higher heat duty;
larger pipes, valves and safety releases;
more electric power for the compressor to recycle the gas and to overcome the pressure drop of the system; and
more heat losses due to the need to reheat and cool much larger amount of gas.

The recycle rate for a given overall COx conversion can be somewhat reduced by minimizing the inerts and excess H₂ in the feed syngas. As a result, methanol plant designers of state of the art syngas-to-methanol processes increase the upstream costs of syngas generation by utilizing high purity feed stock containing low inert species, and adjusting the hydrogen to COx ratio such that hydrogen excess is only 1-3% greater than the stoichiometric requirement. For an Auto Thermal reformer (ATR), the oxygen being used is low in N₂ making it more expensive.

All the costs associated with the issues described above make methanol plants very expensive. To lower the cost per gallon of product and to enjoy economy of scale, the trend in the syngas-to-methanol industry is to build ever larger mega-plants.

Therefore, there remains a need for a syngas-to-methanol process that avoids many of the drawbacks mentioned above, including the need to use low inerts natural gas as a feedstock to the reformer, the need to carefully adjust the composition of the syngas, and the need to recycle and process large quantities of gas in the methanol plant.

### SUMMARY OF THE INVENTION

Accordingly, the current invention allows for economically attractive, relative small methanol plants built modularly and supplying methanol in locations where currently, due to high cost, methanol has to be transported from far away mega plants. An aspect of the present invention provides a once through methanol process using multi-stage single reactor vessel in jointly cooled shell to convert syngas to methanol. The overall conversion efficiency depends on the number of the stages that are becoming smaller as the reaction progresses. There is no recycle compressor. The reactor is relatively small and can handle syngas containing nitrogen and excess hydrogen with only small impact on capital costs and thus high impact on operating costs and total cost of methanol.

Another aspect of the present invention provides a reactor system for producing methanol from syngas. The system contains a reactor and associated condensers for separating syngas from methanol and water. The reactor is a shell and tube reactor that is divided at its top and bottom heads into several vertical, isolated compartments. Each compartment in the top head is associated with tubes and a compartment in the bottom head, which constitute a stage of the reactor. A stream exiting each stage is fluidly connected to a condenser for separating methanol and water from he residual syngas of that stage. The residual syngas is then fed to the next stage in the reactor.

Other aspects of the invention, including apparatuses, devices, processes, and the like which constitute part of the invention, will become more apparent upon reading the following detailed description.

### BRIEF DESCRIPTION OF THE DRAWINGS

The accompanying drawings are incorporated in and constitute a part of the specification. The drawings, together with the general description given above and the detailed description, serve to explain the principles of the invention. In such drawings:

FIG. 1 shows a schematic of an embodiment of the multi-stage, once through reactor of the present invention;

FIG. 2 shows a cut-away top view of the top head of the reactor; and

FIG. 3 shows a cut-away bottom view of the bottom head of the reactor.

### DETAILED DESCRIPTION

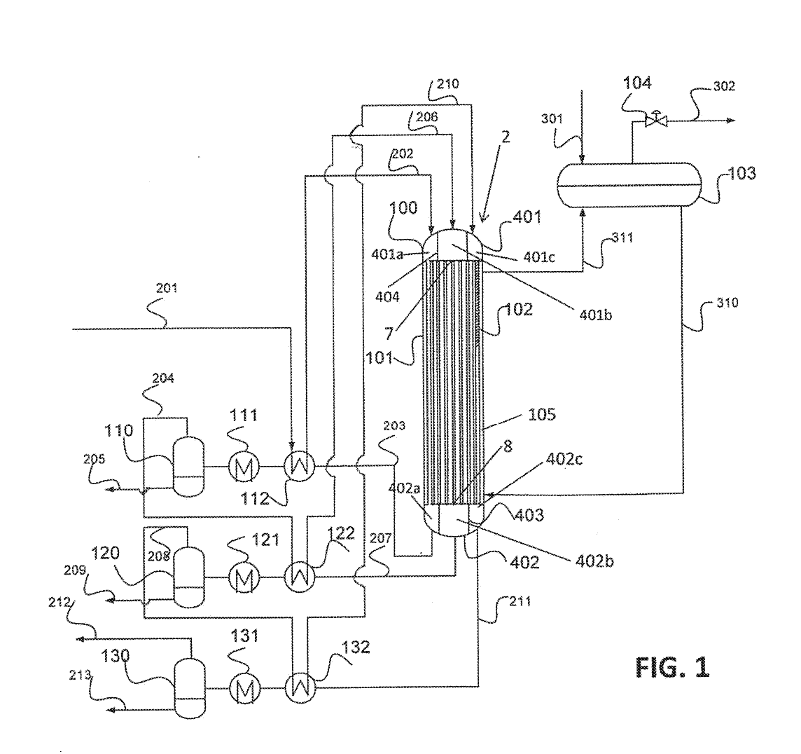

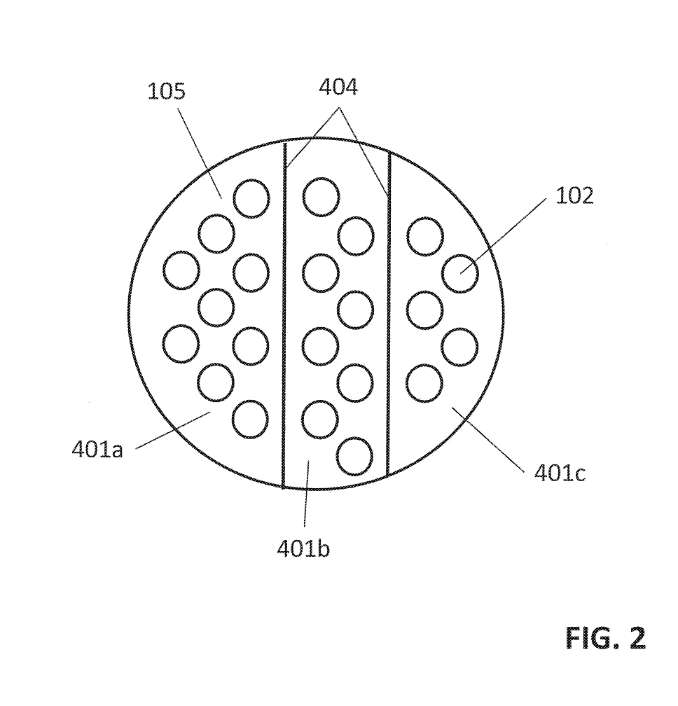

Referring to FIGS. 1-2, the reactor of the present invention is a shell and tube reactor 2, which generally contains a vessel 100 containing a plurality of tubes 102 extending axially inside the vessel 100. The tubes 102 extend a majority of the length of the vessel 100 and are designed to carry out syngas-to-methanol reactions in its lumen, while the shell side 105 of the reactor 2 is designed to allow a coolant to flow therethrough. The reactor 2 also contains a top head 401 (the top head 401 is divided into several compartments, e.g. 401a, 401b, 401c as explained below) for feeding reactants into the tubes 102 and a bottom head 402 (the bottom head 402 is divided into several compartments, e.g. 402a, 402b, 402c as explained below) for collecting reaction product(s) and unreacted reactants from the tubes 102. A top tube sheet 7 separates the top head 401 from the shell side 105 of the reactor, such that fluid communication between the top head 401 and the lumen of the tubes 102 is preserved, but fluid communication between top head 401 and the shell side 105 is obstructed. Thus, reactants entering the top head 401 flows into the tubes 102, but not the shell side 105. Likewise, the coolant on the shell side 105 is also prevented from entering the top head 401 by the top tube sheet 7. A bottom tube sheet 8 also similarly separates the bottom head 402 from the shell side 105. Here, the reactant mixture inside the tubes can enter the bottom head 402, but coolant from the shell side 105 cannot.

The top header 401 is divided into several isolated compartments 401a, 401b, 401c by dividing walls 404 which is place approximately perpendicular to the top tube sheet 7. The compartments 401a, 401b, 401c are fluidly isolated from each other so that gas or liquid in one compartment cannot pass into adjacent compartment(s). As such, the gas or liquid in a compartment can only enter the tubes exposed to that particular compartment. For example, gas or liquid in compartment 401a can only enter tubes 102 that are exposed to compartment 401a. Each of the compartments 401a, 401b, 401c contains an inlet that allows reactants to enter the compartment.

The bottom head 402 is also divided into several isolated compartments 402a, 402b, 402c by dividing walls 403 which is place approximately perpendicular to the bottom tube sheet 8. The dividing walls 403 are positioned so that the isolated compartments 402a, 402b, 402c mirrors their respective isolated compartments 401a, 401b, 401c of the top head 401. That way, gas or liquid that enters compartment 401a can only exit into its respective compartment 402a, but not 402b or 402c. Likewise, gas or liquid that enters compartment 401b can only exit into compartment 402b, but not 402a or 402c, etc. Each of the compartments 402a, 402b, 402c contains an outlet that allows the reaction mixture in the compartment to be removed from that compartment.

Between the heads 401, 402 is the shell and tubes section 101 of the reactor 2. The tubes fluidly connects compartments 401a, 401b, 401c of the top head 401 to their respective compartments 402a, 402b, 402c of the bottom head 402. The respective compartments (401a and 402a, 401b and 402b, or 401c and 402c) are connected by a defined number of tubes to define a stage of the reactor 2. Thus, respective compartments and tubes are referred to as being “associated” to form a stage in the reactor. The tubes 102 are designed to conduct the syngas-to-methanol reactions and may contain catalysts therein. Syngas typically contains CO, CO₂ and H₂ as the active species and CH₄ and N₂ as inert species. To enhance the syngas-to-methanol reactions, the catalyst used may be, e.g., a metal catalyst, typically copper/zinc based catalyst

Although the drawings show three isolated compartments in each of the top and bottom heads 401, 402, the present invention may have more or less isolated compartments. In exemplary embodiments, the present invention may contain 2 to 10 compartments, preferably 3-6 compartments. Each of the isolated compartments may be designed to be associated with the same number of tubes. For example, each of compartments 401a, 401b, 401c (and thus compartments 402a, 402b, 402c) may be associated with ten tubes. But in certain embodiments, it may be advantageous to design the different isolated compartments to be associated with different number of tubes 102. For example, compartment 401a (and thus compartment 402a) may be associated with fifteen tubes, while compartment 401b (and this compartment 402b) may be associated with ten tubes. Thus, each stage of the reactor may contain a different number of tubes 102. Because the amount of gas handled by each subsequent stage of the reactor is lower than the previous stage, it may be advantageous to reduce the number of tubes 18 in each of the subsequent stages.

Each of the isolated compartments 401a, 401b, 401c of the top head 401 and its associated tubes and bottom head compartment 402a, 402b, or 402c form a stage in the reactor to conduct the syngas-to-methanol reactions. A first stage takes in syngas, e.g. from a reformer or a gasifier, and reacts the syngas in the first stage tubes. The reaction mixture from the first stage is then fed to a first separator 110 which separates the reaction mixture into a liquid containing methanol and water, and a gas containing unreacted syngas. The unreacted syngas is then fed to a second stage for further reaction. The reaction mixture exiting the second stage is fed to a second separator 120 which separates the reaction mixture into a liquid containing methanol and water, and a gas containing unreacted syngas. The unreacted syngas from the second stage may then be fed into a third stage for further reaction. The process may continue for as many stages as the reactor design allows.

FIG. 1 illustrates the once-through three-stage process in a single methanol reactor 2. The vessel 100 is a pressure vessel containing boiling water on the shell side 105 for cooling of the syngas-to-methanol reaction occurring in the tubes 102. The cooling of the reaction is accomplished by adding water 301 to the steam drum 103. The water naturally or mechanically circulates through stream 310 to the bottom part of the reactor 2 and enters the shell side 105. The heat generated by the reaction in the tubes 102 boils the water on the shell side 105 to generate two phase flow at a pressure controlled by the back pressure control valve 104. The pressure control is designed to maintain the temperature of the boiling water in the shell side 105 in the range of about 200 to about 280° C., preferably about 220 to about 260° C. The medium pressure steam in stream 302 discharges from the steam drum through the pressure control valve 104 and may be used for methanol distillation or other applications.

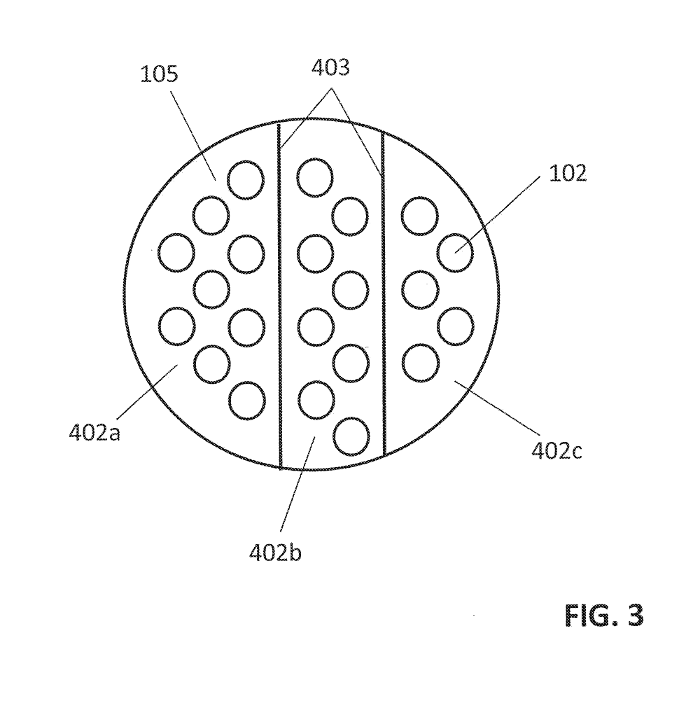

The top and bottom heads 401, 402 of the reactor 2 are a similar to each other, as best illustrated in FIGS. 2 and 3. The top head 401 and the bottom head 402 are internally divided by plates 404 and 403, respectively, to compartments which are gas tight. Gas entering the top head, stream 202 for example, can only flow downwards through the tubes 102 which are opened to a top compartment 401a and to a bottom compartment 402a. As the gas flows through the catalyst filled tubes, it reacts and generates heat. The heat is removed by boiling water in the shell side 105 in contact with those tubes. The gas comes out of the tubes 102 at the bottom and is confined in the compartment 402a. In an exemplary embodiment, the pressure in the shell and tube side of the reactor is in the range of about 30 to about 120 bars, but the pressure drop across the dividing wall 404 or 403 is typically less than about 0.5 bars so that the plates 404,403 need not be heavily fortified.

Stream 201 contains pressurized syngas, preferably at ambient or higher temperature. The syngas contains CO, CO₂ and H₂, in addition to inerts, such as N₂ and CH₄. Without any modification to the syngas from the syngas generator, when an Auto-Thermal Reformer (ATR) is used as the syngas generator, the hydrogen concentration is typically lower than stoichiometricly required; therefore, it is preferred that the syngas from the generator is adjusted by removing CO₂ before being fed into the reactor. Alternatively, when a steam methane reformer (SMR) is used to generate syngas, the hydrogen concentration is about 20 to about 33% in excess of the stoichiometric requirement. In that case, the excess hydrogen may be removed upstream or downstream of the methanol system. That excess hydrogen may be used as fuel, for example. The feed syngas may be preheated, preferably to a range of about 170 to about 250° C. in a gas-to-gas heat exchanger 112 by the hotter stream 203; and the heated syngas in stream 202 flows into the first stage compartment 401a in the top head 401 and further into the tubes 102 of the first stage. The first stage handles the entire volume of the feed gas to produce methanol and water based on the syngas-to-methanol reactions.

The gas exiting the first stage bottom compartment 402a, in stream 203, is cooled in the gas-gas heat exchanger 112 and further in heat exchanger 111 to a condensing temperature. The heat exchanger 111 may use cooling water or ambient air cooling. Methanol and water condense from the gas and are separated in separator 110. The methanol and water liquid stream 205 from separator 110 may be sent to a crude methanol storage tank via stream 205, while the remaining gas (unreacted syngas) is sent to the next stage for further reaction.

After separating the condensing products, methanol and water, the remaining gas, in stream 204, may be effectively used to produce additional methanol in a second stage. Accordingly, the gas in stream 204 is heated in the gas-to-gas heat exchanger 122 and the heated stream 206 is sent to the top compartment 401b of the second stage. Additional syngas-to-methanol reactions takes place in the second stage and the resultant stream 207, exiting compartment 402b, is cooled in heat exchanger 122 and heat exchanger 121 (heat exchangers 122 and 121 operates similarly to heat exchangers 112 and 111, respectively, as described above). Methanol and water produced in the second stage are condensed from the gas and separated in separator 120. The methanol and water liquid stream 209 from separator 120 may be sent to the crude methanol storage tank, while the remaining gas is sent to the next stage for further reaction.

After separating the condensing products, methanol and water, the remaining gas from the second stage can be effectively converted to produce additional methanol in a third stage. Stream 208 is heated in the gas-to-gas heat exchanger 132 and the heated stream 210 is sent to compartment 401c of the third stage. Additional syngas-to-methanol reaction then takes place in this third stage and the resultant stream 211, exiting compartment 402c, is cooled in heat exchanger 132 and heat exchanger 131 (heat exchangers 132 and 131 operates similarly to heat exchangers 112 and 111, respectively, as described above). Methanol and water are condensed and separated from the gas in separator 130. The methanol and water stream 213 from the separator 130 may be sent to the crude methanol storage tank, while the remaining gas in stream 212 may be further used as fuel in a reformer or for other purposes.

During the process, the first stage of the reactor 2 handles the entire feed; the second stage handles a smaller residual syngas volume remaining after removing the condensate in separator 110; and the third stage handles even a smaller gas volume after removing the condensate in separator 120. Although three stages are discussed above, additional stages may be added to the process with each stage handling a smaller gas volume than the stage immediately upstream.

In an exemplary embodiment, the methanol in the condensate product of each of the reactor stages may be further purified, e.g. by distillation. In that case, it is preferred that the product is fed to a stage of the distillation column that has a concentration that best matches the methanol concentration of the product. For example, if the methanol product in stream 205 has a concentration of about 90%, then it is preferably fed to a stage of the distillation column having a concentration of approximately 90% (or one that best matches 90%). Further, if the methanol product in stream 209 has a concentration of about 60%, then it is preferably fed to a stage of the distillation column having a concentration of approximately 60% (or one that best matches 60%). Additionally, if the methanol product in stream 213 has a concentration of about 50%, then it is preferably fed to a stage of the distillation column having a concentration of approximately 50% (or one that best matches 50%). The same may be repeated for any subsequent stage(s) of the reactor.

Without further description, it is believed that one of ordinary skill in the art can, using the preceding description and the following illustrative example, make and utilize the apparatuses of the present invention and practice the methods. The following example is given to illustrate the present invention. It should be understood that the invention is not to be limited to the specific conditions or details described in the example.

### EXAMPLE

Table 1 illustrates the change in flow rate and composition in each stage of a five-stage, once-through reactor of the present invention. The syngas feed was 100 Kgmole/h. The first stage handled the largest volume and converts the most COx into methanol and water. The results in Table 1 are for illustration only, and actual reactor performance depends on the operating pressure and temperature of the reactor, the number, diameter and length of tubes in each stage, and the type and activity of the catalyst being used. More stages result in higher COx conversion and more methanol production and the result is only limited by economic considerations.

TABLE 1
Flow rate, composition, and COx conversion in each methanol
reactor stage per 100 Kgmole/h of feed rate.

| | | Stage 1 | Stage 2 | Stage 3 | Stage 4 | Stage 5 |
|---|---|---|---|---|---|---|
| | Feed | Outlet | Outlet | Outlet | Outlet | Outlet |
| H2, kgmole/h | 74.20 | 43.08 | 33.22 | 27.21 | 23.44 | 21.42 |
| CO, Kgmole/h | 15.77 | 1.58 | 0.36 | 0.21 | 0.14 | 0.09 |
| CO2, | 8.34 | 6.89 | 4.32 | 2.39 | 1.17 | 0.53 |
| Kgmole/h | | | | | | |
| CH4, | 1.51 | 1.51 | 1.51 | 1.51 | 1.51 | 1.51 |
| Kgmole/h | | | | | | |
| N2, Kgmole/h | 0.17 | 0.17 | 0.17 | 0.17 | 0.17 | 0.17 |
| Total, | 100.00 | 53.22 | 39.58 | 31.49 | 26.43 | 23.72 |
| Kgmole/h | | | | | | |
| COx | 0.00 | 64.89 | 80.61 | 89.24 | 94.58 | 97.44 |
| conversion | | | | | | |
| (mole %) | | | | | | |
| Methanol | | 15.64 | 3.79 | 2.08 | 1.29 | 0.69 |
| produced, | | | | | | |
| Kgmole/h | | | | | | |
| Water | | 1.45 | 2.57 | 1.93 | 1.22 | 0.64 |
| produced, | | | | | | |
| Kgmole/h | | | | | | |
| Methanol | | 92% | 60% | 52% | 51% | 52% |
| fraction | | | | | | |
| (mole %) | | | | | | |

The total flow through the reactor (the combined feed to the five stages) was about 250 Kgmole/h. For comparison, in a conventional boiling water cooled methanol reactor using similar syngas and operating under similarly conditions, a recycle rate of 5-6 moles of recycle gas is required for each one mole of feed gas. That means that the conventional reactor has to be designed to handle 600-700 Kgmole/h of gas per 100 Kgmole/h of feed gas. That is much greater than the 250 Kgmole/h in the reactor of the current invention. In addition, a recycle compressor and electric power to recycle 500-600 Kgmole/h of syngas has to be used. The multi-stage, once through, single reactor of the present invention does not need to recycle the syngas. As such, no compressor and no power usage is needed for recycling.

The table demonstrated an advantage in segregating the methanol/water condensates to economize in the purification of the product to produce high purity methanol. Methanol was highly concentrated in the first separator product and decreased in subsequent stages. The methanol may be further purified in a subsequent process, e.g. a distillation column. In that case, the separator product of the different reactor stages may be introduced to different separation stages of a distillation column that match the methanol concentrations of the separator products. That matching of concentration between the separator product and the distillation stage economizes the energy needs and capital costs of the subsequent purification step.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. The specification describes specific examples to accomplish a more general goal that may be accomplished in another way. Accordingly, it is intended that the invention be limited only to the extent required by the appended claims and the applicable rules of law.

What is claimed is:

1. A reactor system comprising a vessel having
a. a plurality of tubes extending axially inside the vessel defining a tube side and a shell side;
b. a top head; and
c. a bottom head,
wherein the top head contains at least one vertical diving wall dividing the top head into at least a first compartment that is isolated from a second compartment, and the bottom head contains at least at least one vertical diving wall diving the bottom head into at least a first compartment that is isolated from a second compartment, the compartments of the bottom head mirroring the compartments of the top head,
wherein each of the isolated portions of the top head contains its own inlet, and each of the isolated portions of the bottom head contains its own outlet, and
wherein the outlet of the first portion of the bottom head is fluidly connected to the inlet of the second portion of the top head.

2. The reactor system of claim 1, wherein each of the top and bottom heads contains two to ten compartments.

3. The reactor system of claim 1, wherein a separator is placed between the outlet of the first compartment of the bottom head and the inlet of the second compartment of the top head.

4. The reactor system of claim 3, wherein the separator is a condenser.

5. The reactor system of claim 1, wherein the tubes are filled with a catalyst for converting synthesis gas to methanol.

6. The reactor system of claim 1, wherein the shell side contains boiling water.

7. The reactor system of claim 6, wherein the boiling water is provided by a steam drum.

8. A process for synthesis of methanol from synthesis gas comprising the steps of
a. providing the reactor system of claim 1;
b. feeding synthesis gas into the first compartment of the top head;
c. reacting the synthesis gas in the tubes associated with the first compartment of the top head to obtain a first reaction mixture, while saturated coolant circulate on the shell side of the reactor in a direction countercurrent to flow in the tubes;
d. separating the first reaction mixture in a first separator to obtain a first product and a first gas, wherein the first product contains methanol and water, and the first gas contains unreacted synthesis gas;
e. feeding the first gas into the second compartment of the top head;
f. reacting the first gas in the tubes associated with the second compartment of the top head to obtain a second reaction mixture, while saturated coolant circulate on the shell side of the reactor in a direction countercurrent to flow in the tubes; and
g. separating the second reaction mixture in a second separator to obtain a second product and a second gas, wherein the second product contains methanol and water, and the second gas contains unreacted synthesis gas.

9. The process of claim 8, wherein the coolant is water or air.

10. The process of claim 8, wherein the first and second products are stored.

11. The process of claim 8, wherein each of the top and bottom heads contains two to ten compartments.

12. The process of claim 8, wherein steps d and g occurs by phase separation.

13. The process of claim 8, wherein steps d and g are accomplished by cooling the first and second reaction mixtures.

14. The process of claim 8, wherein the wherein the tubes are filled with a catalyst for converting synthesis gas to methanol.

15. The process of claim 8, further comprising the steps of
a. feeding the second gas into a third compartment of the top head;
b. reacting the second gas in the tubes associated with the third compartment of the top head to obtain a third reaction mixture, while saturated coolant circulate on the shell side of the reactor in a direction countercurrent to flow in the tubes; and
c. separating the third reaction mixture in a third separator to obtain a third product and a third gas, wherein the third product contains methanol and water, and the third gas contains unreacted synthesis gas.

16. The process of claim 8, further comprising the step of purifying the methanol.

17. The process of claim 16, wherein the purifying step takes place in a distillation column.

18. The process of claim 17, wherein the first product is fed to a stage of the distillation column having a methanol concentration matching that of the first product, and the second product is fed to another stage of the distillation column having a methanol concentration matching that of the second product.
